(19) United States
(12) Patent Application Publication
(10) Pub. No.: US 2007/0142795 A1
(43) Pub. Date: Jun. 21, 2007

(54) Food scented personal hygiene products

(75) Inventors: Bliss E. Cohen, Appleton, WI (US); Daryl S. Bell, Appleton, WI (US); Garry Roland Woltman, Appleton, WI (US); Katherine Carol Wheeler, Menasha, WI (US)

Correspondence Address: SENNIGER POWERS (KCC), ONE METROPOLITAN SQUARE, 16TH FLOOR, ST LOUIS, MO, 63102, US

(73) Assignee: Kimberly-Clark Worldwide, Inc., Neenah, WI (US)

(21) Appl. No.: 11/300,858

(22) Filed: Dec. 15, 2005

(51) Int. Cl. A61F 13/15

(52) U.S. Cl. 604/359; 604/385.02

(57) ABSTRACT

The present disclosure generally relates to absorbent products, such as absorbent articles and packages of absorbent articles, that have a food scent. More preferably, the absorbent products have a chocolate scent. Alternately or in addition, the absorbent products may comprise a food related reward. Also provided are methods for rewarding a consumer for the purchase of such absorbent products.

### BACKGROUND OF DISCLOSURE

The present disclosure generally relates to absorbent products, such as absorbent articles and packages of absorbent articles, that have a food scent. More particularly, the absorbent products have a chocolate scent. Alternately or in addition, the absorbent products may comprise a food related reward. Also provided are methods for rewarding a consumer for the purchase of such absorbent products.

Chocolate has been known in many forms for many years. Traditional forms of chocolate include dark, light, white, and milk chocolates which may be used as a snack item or confection, or for coating other food items. More recently, chocolate has been utilized not only as a flavor but in chip or chunk form as an additive in baked goods and flour confections such as cookies and the like.

Chocolate has an almost universal appeal simply for its flavor. Although considered pleasurable to eat, chocolate is typically perceived as lacking desired health benefits, and consequently is seen as a “forbidden food,” that should be eaten only as a special treat.

Many people associate “forbidden foods” such as salty snacks and sweet snacks like chocolate with comfort. Such snacks are often eaten in times of stress or during negative moods, such as boredom, tension, anger, depression, and tiredness. Chocolate, in particular, is seen as a comfort food.

Studies have found that women crave certain foods during menstruation. Particularly desired are foods that are considered pleasurable, but unhealthy or “bad for you,” such as chocolate. Menstruation is seen by many women as an excuse to eat, and as a time when it is acceptable to eat forbidden foods, such as chocolate.

### SUMMARY OF THE DISCLOSURE

The present disclosure generally relates to absorbent products, such as absorbent articles and packages of absorbent articles, that have a food scent. More particularly, the absorbent products have a chocolate scent. Alternately or in addition, the absorbent products may comprise a food related reward. Also provided are methods for rewarding a consumer for the purchase of such absorbent products.

In one embodiment, the present disclosure provides a package of absorbent articles. The package comprises a plurality of individual absorbent articles; a packaging comprising a pocket having a hollow interior for receiving said plurality of absorbent articles and an opening mechanism, which provides access to said absorbent articles; and a food scent producing agent. The food scent produced by the food scent producing agent is selected from the group consisting of a chocolate scent and a scent associated with a salty snack.

In another embodiment, the present disclosure provides a package of absorbent articles. The package comprises a plurality of absorbent articles; a packaging comprising a pocket having a hollow interior for receiving said plurality of absorbent articles and an opening mechanism, which provides access to said absorbent articles; and a food benefit coupon.

Also provided is an absorbent article. The absorbent article comprises an absorbent structure; and a food scent producing agent. The food scent produced by the food scent producing agent is selected from the group consisting of a chocolate scent and a scent associated with a salty snack.

The present disclosure also provides a method for rewarding a consumer for a purchase. The method comprises determining when the consumer purchases an absorbent product; and authorizing a third party to distribute a food related reward to the consumer when the determining indicates that the absorbent product has been purchased by the consumer.

Also provided is a method for rewarding a consumer for a purchase. The method comprises determining when the consumer purchases an absorbent product; providing to the consumer a plurality of choices of food related rewards when the determining indicates that the absorbent product has been purchased by the consumer; receiving from the consumer a selection of at least one of the food related rewards from the plurality of choices; and distributing the selection of food related reward to the consumer.

In another embodiment, the present disclosure provides a system for distributing a reward to a consumer. The system comprises means for monitoring consumer purchases to determine when the consumer purchases an absorbent product; means for signaling a third party when the monitoring means indicate that the absorbent product is purchased by the consumer; and means for distributing a food related reward to the consumer in response to the purchase of the absorbent product by the consumer.

Also provided is a system for distributing a reward to a consumer. The system comprises means for monitoring when the consumer purchases an absorbent product; means for maintaining a consumer database containing information on monitored purchases by the consumer; means for receiving from the consumer a selection of a food related reward from a plurality of choices; and means for distributing the selection of the food related reward to the consumer as function of the information in the consumer database.

Other objects and features will be in part apparent and in part pointed out hereinafter.

### BRIEF DESCRIPTION OF THE DRAWINGS

The various features, aspects and advantages of the present disclosure will become better understood with reference to the following description, appended claims and accompanying drawings.

FIG. 1 is a top plan of a feminine napkin of the present disclosure.

FIG. 2 is a bottom plan of the napkin.

FIG. 3 is a section taken in the plane of line 3-3 of FIG. 1.

FIG. 4 is a top plan of another feminine napkin of the present disclosure having wings.

FIG. 5 is a bottom plan of the napkin shown in FIG. 4.

FIG. 6 is a section taken in the plane of line 6-6 of FIG. 4.

Corresponding reference characters indicate corresponding parts throughout the drawings.

### DETAILED DESCRIPTION OF THE DISCLOSURE

The present disclosure generally relates to absorbent products, such as absorbent articles and packages of absorbent articles, that have a food scent. More particularly, the absorbent products have a chocolate scent. The absorbent products may additionally or alternatively comprise a food related reward, such as a food item, a food scented item, a food benefit coupon, and the like, and combinations thereof. Also provided are methods for rewarding a consumer for the purchase of such absorbent products.

The present disclosure is discussed primarily in combination with feminine hygiene products such as feminine napkins, panty liners, tampons, and interlabial pads. However, it will be readily apparent to one skilled in the art based on the disclosure that the products and methods described herein can also be used in combination with numerous other absorbent articles. As used herein, the phrase “absorbent article” generally refers to devices which absorb and contain body fluids, and more specifically, refers to devices which are placed against or near the skin to absorb and contain the various fluids discharged from the body. Examples of other absorbent articles include absorbent articles intended for personal wear, such as diapers; incontinence products; feminine hygiene products, such as feminine napkins, panty liners, tampons, and interlabial pads; other personal garments; and the like.

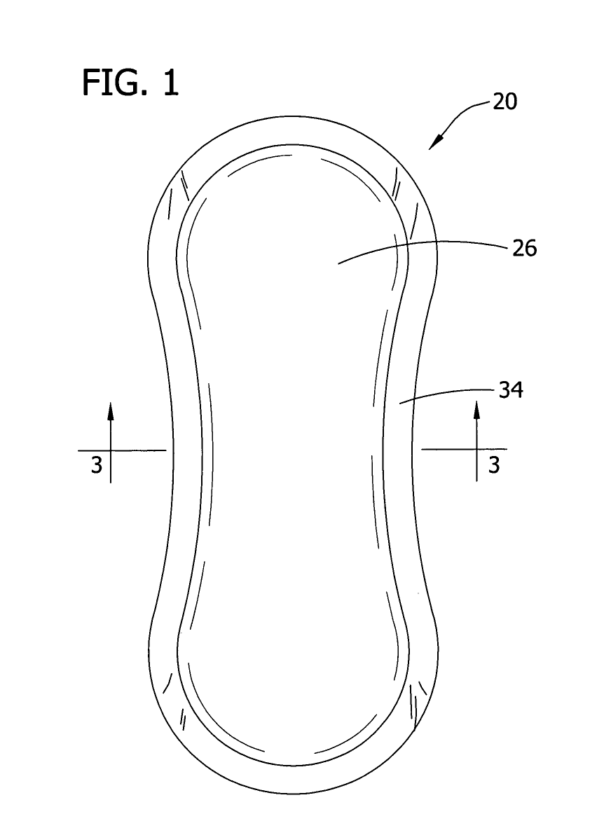

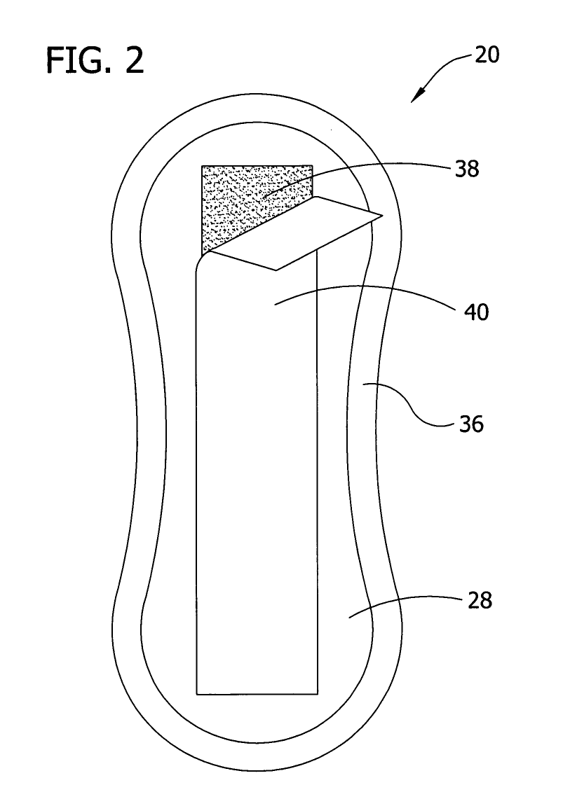

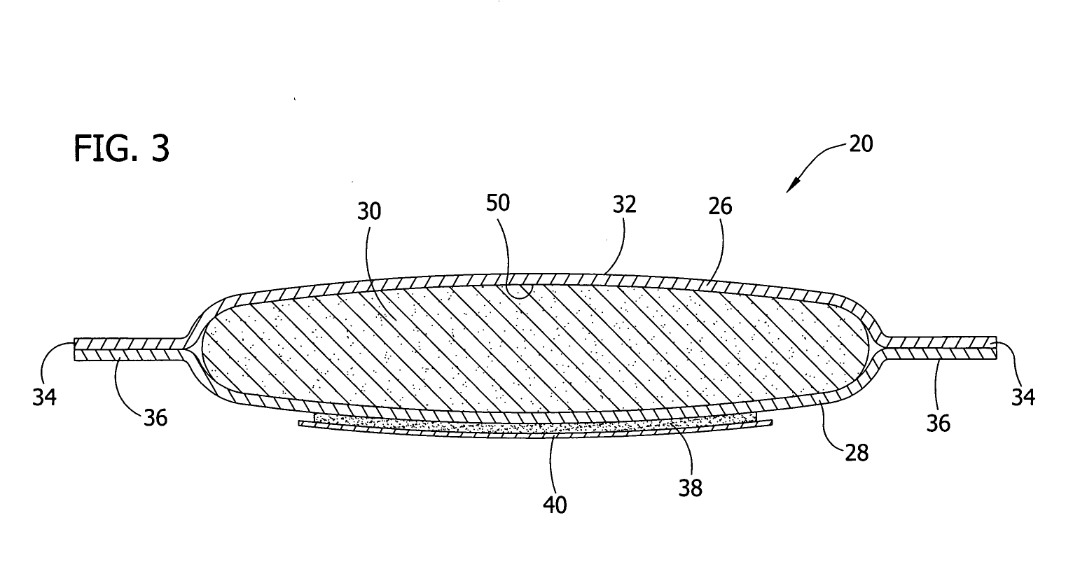

Referring now to the drawings and in particular to FIGS. 1-6, a representative article, such as the representatively shown feminine care article, is shown. The feminine care article can, for example, be a feminine care pad or sanitary napkin, which is indicated generally at 20. As illustrated in FIGS. 1-3, the napkin 20 can include a topsheet 26, a backsheet 28, and an absorbent core 30 positioned between the topsheet and the backsheet. The topsheet 26 may include a layer constructed of any operative material, and may be a composite material. For example, the topsheet 26 can include a woven fabric, a nonwoven fabric, a polymer film, a film-fabric laminate or the like, as well as combinations thereof. As used herein, the term “nonwoven” refers to a fabric web that has a structure of individual fibers or filaments which are interlaid, but not in an identifiable repeating manner. Examples of a nonwoven fabric include spunbond fabric, meltblown fabric, coform fabric, a carded web, a bonded-carded-web, a bicomponent spunbond fabric or the like as well as combinations thereof.

For example, the topsheet 26 can include a woven fabric, a nonwoven fabric, a polymeric film that has been configured to be operatively liquid-permeable, or the like, as well as combinations thereof. Other examples of suitable materials for constructing the topsheet 26 can include rayon, bonded carded webs of polyester, polypropylene, polyethylene, nylon, or other heat-bondable fibers, polyolefins, such as copolymers of polypropylene and polyethylene, linear low-density polyethylene, aliphatic esters such as polylactic acid, finely perforated film webs, net materials, and the like, as well as combinations thereof.

A more particular example of a suitable topsheet 26 material can include a bonded-carded-web composed of polypropylene and polyethylene, such as has been used as a cover stock for KOTEX brand pantiliners, and has been obtainable from Vliesstoffwerk Christian Heinrich Sandler GmbH & Co. KG, a business having an address at Postfach 1144, D95120 Schwarzenbach/Saale, Germany. Other examples of suitable materials are composite materials of a polymer and a nonwoven fabric material. The composite materials are typically in the form of integral sheets generally formed by the extrusion of a polymer onto a web of spunbond material. As used herein, the terms “spunbond” or “spunbonded fiber” refer to fibers which are formed by extruding filaments of molten thermoplastic material from a plurality of fine, usually circular, capillaries of a spinneret, and then rapidly reducing the diameter of the extruded filaments.

In a desired arrangement, the topsheet 26 can be configured to be operatively liquid-permeable with regard to the liquids that the napkin 20 is intended to absorb or otherwise handle. The operative liquid-permeability may, for example be provided by a plurality of pores, perforations, apertures or other openings, as well as combinations thereof, that are present or formed in the topsheet. The apertures or other openings can help increase the rate at which bodily liquids can move through the thickness of the topsheet 26 and penetrate into the other components of the napkin 20 (e.g., the absorbent core 30). The selected arrangement of liquid-permeability is desirably present at least on an operative portion of the topsheet 26 that is appointed for placement on the body-side of the napkin 20.

The topsheet 26 can provide comfort and conformability, and can function to direct bodily exudates away from the body and toward the absorbent core 30. In a desired feature, the topsheet 26 can be configured to retain little or no liquid in its structure, and can be configured to provide a relatively comfortable and non-irritating bodyside surface 32 (broadly, an “engagement surface”) next to the tissue layer (i.e., skin) of a female wearer. The topsheet 26 can be constructed of any material easily penetrated by bodily fluids that contact the surface of the topsheet.

The topsheet 26 can also have at least a portion of its bodyside surface 32 treated with a surfactant to render the topsheet more hydrophilic. The surfactant can permit arriving bodily liquids to more readily penetrate the topsheet 26. The surfactant may also diminish the likelihood that the arriving bodily fluids, such as menstrual fluid, will flow off the topsheet 26 rather than penetrate through the topsheet into other components of the napkin 20 (e.g., the absorbent core 30). In a particular configuration, the surfactant can be substantially evenly distributed across at least a portion of the upper, bodyside surface 32 of the topsheet 26 that overlays the upper, bodyside surface of the absorbent core 30.

The topsheet 26 may be maintained in secured relation with the absorbent core 30 by bonding all or a portion of the adjacent surfaces to one another. A variety of bonding articles known to one of skill in the art may be utilized to achieve any such secured relation. Examples of such articles include, but are not limited to, the application of adhesives in a variety of patterns between the two adjoining surfaces, entangling at least portions of the adjacent surface of the absorbent with portions of the adjacent surface of the topsheet 26, or fusing at least portions of the adjacent surface of the topsheet to portions of the adjacent surface of the absorbent core 30.

The topsheet 26 typically extends over the upper, bodyside surface of the absorbent core 30, but can alternatively extend around the napkin 20 to partially or entirely, surround or enclose the absorbent core 30. Alternatively, the topsheet 26 and the backsheet 28 can have peripheral margins 34, 36 that extend outwardly beyond the terminal, peripheral edges of the absorbent core 30, and the extending margins can be joined together to partially or entirely, surround or enclose the absorbent core.

The backsheet 28 may include a layer constructed of any operative material, and may or may not have a selected level of liquid-permeability or liquid-impermeability, as desired. In a particular configuration, the backsheet 28 may be configured to provide an operatively liquid-impermeable backsheet structure. The backsheet 28 may, for example, include a polymeric film, a woven fabric, a nonwoven fabric or the like, as well as combinations or composites thereof. For example, the backsheet 28 may include a polymer film laminated to a woven or nonwoven fabric. In a particular feature, the polymer film can be composed of polyethylene, polypropylene, polyester or the like, as well as combinations thereof. Additionally, the polymer film may be micro-embossed, have a printed design, have a printed message to the consumer, and/or may be at least partially colored. Desirably, the backsheet 28 can operatively permit a sufficient passage of air and moisture vapor out of the napkin 20, particularly out of an absorbent core 30 while blocking the passage of bodily liquids. An example of a suitable backsheet 28 can include a breathable, microporous film, such as a HANJIN Breathable Baffle available from Hanjin Printing, Hanjin P&C Company Limited, a business having offices located in Sahvon-li.Jungan-mvu.Kongiu-City, Chung cheong nam-do, Republic of South Korea. The backsheet 28 material is a breathable film, which is white in color, dimple embossed and contains: 47.78% calcium carbonate, 2.22% TiO₂, and 50% polyethylene.

Bicomponent films or other multi-component films can also be used as backsheet 28 material, as well as woven and/or nonwoven fabrics which have been treated to render them operatively liquid-impermeable. Another suitable backsheet 28 material can include closed cell polyolefin foam. For example, closed cell polyethylene foam may be employed. Still another example of a backsheet 28 material would be a material that is similar to a polyethylene film which is used on commercially sold KOTEX brand pantiliners, and is obtainable from Pliant Corporation, a business having offices located in Schaumburg, Ill., USA.

The structure of the absorbent core 30 can be operatively configured to provide a desired level of absorbency or storage capacity. More particularly, the absorbent body can be configured to hold a liquid, such as urine, menses, complex liquid or the like, as well as combinations thereof. The absorbent core 30 can include a matrix of absorbent fibers and/or absorbent particulate material, and the absorbent fiber can include natural and/or synthetic fiber. Additionally, the absorbent core 30 may include one or more components that can modify menses or intermenstrual liquid.

The absorbent core 30 may also include superabsorbent material. Superabsorbent materials are known to those skilled in the art, and may be in any operative form, such as particulate form. Generally stated, the superabsorbent material can be a water-swellable, generally water-insoluble, hydrogel-forming polymeric absorbent material, which is capable of absorbing at least about 20, desirably about 30, and possibly about 60 times or more its weight in physiological saline (e.g. saline with 0.9 wt % NaCl). The hydrogel-forming polymeric absorbent material may be formed from organic hydrogel-forming polymeric material, which may include natural material such as agar, pectin, and guar gum; modified natural materials such as carboxymethyl cellulose, carboxyethyl cellulose, and hydroxypropyl cellulose; and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyacrylamides, polyvinyl pyridine, and the like. Other suitable hydrogel-forming polymers include hydrolyzed acrylonitrile grafted starch, acrylic acid grafted starch, and isobutylene maleic anhydride copolymers and mixtures thereof.

The hydrogel-forming polymers are preferably lightly crosslinked to render the material substantially water insoluble. Crosslinking may, for example, be by irradiation or covalent, ionic, Van der Waals, or hydrogen bonding. Suitable materials are available from various commercial vendors such as The Dow Chemical Company and Stockhausen, Inc. The superabsorbent material may desirably be included in an appointed storage or retention portion of the absorbent system, and may optionally be employed in other components or portions of the napkin 20.

The absorbent core 30 can be arranged in any operative shape and/or design. For example, the absorbent core 30 may comprise a composite structure (not shown) having a selected plurality of strata or layers or a unitary structure. Moreover, the material of the absorbent core 30 can be selected and configured to provide desired liquid-intake properties to quickly absorb and pull liquid away from the body. Accordingly, the absorbent core 30 can provide the function of liquid intake and can also provide the functions of liquid distribution, spreading, liquid retention, and shape maintenance. The absorbent core 30 may include natural fibers, synthetic fibers, superabsorbent materials, a woven fabric; a nonwoven fabric; a wet-laid fibrous web; a substantially unbonded airlaid fibrous web; an operatively bonded, stabilized-airlaid fibrous web; or the like, as well as combinations thereof. Additionally, the absorbent core 30 may include one or more components that can modify menses or intermenstrual liquid.

In a particular arrangement, the absorbent core 30 can be a thermally-bonded, stabilized airlaid fibrous web available from Concert Fabrication (Concert code 225.1021), a business having offices located in Gatineaux, Quebec, Canada (e.g. Concert code 225.1021). The absorbent core 30 may also be a similar, stabilized airlaid fibrous web available from Buckeye Technologies, Inc., a business having offices located in Memphis, Tenn., U.S.A.

Additionally, a selected configuration of garment adhesive 38, such as the illustrated strip regions, may be distributed onto the garment-side surface of the napkin 20 to help secure the napkin to the undergarment (not shown). Typically, the garment adhesive 38 can be distributed over the garment-side surface of the backsheet 28, and one or more layers or sheets of release material 40 can be removably placed over the garment adhesive 38 during storage prior to use.

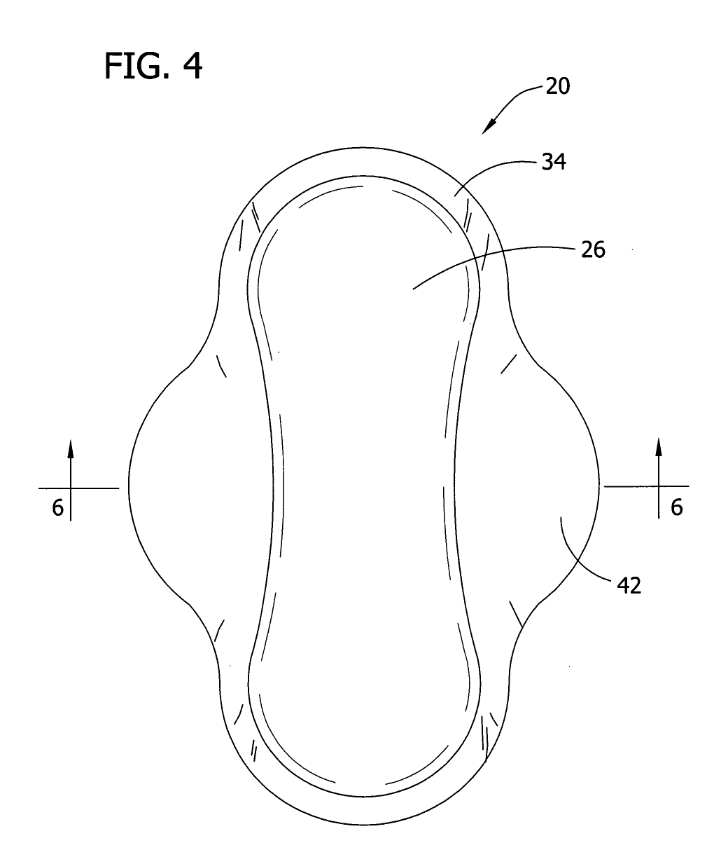

As illustrated in FIGS. 4-6, the napkin 20 can include a system of wing portions 42 which can be integrally connected to appointed sections of the napkin. After placing the napkin 20 in the undergarment, the wings 42 can be operatively wrapped and secured around the side edges of the undergarment to help hold the napkin in place as is know in the art. The wing portions 42 can be separately provided members that are subsequently attached or otherwise operatively joined to intermediate portions of the napkin 20.

In other configurations, the wing portions 42 can be unitarily formed with one or more components of the napkin 20. Either or both wing portions 42 may be formed from a corresponding, operative extension of the material employed to form the topsheet 26. Alternatively, either or both wing portions 42 may be formed from a corresponding, operative extension of the material employed to form the backsheet 28, or formed from a corresponding, operative combination of the topsheet 26 and backsheet materials.

The wing portions 42 can have any operative construction, and can include a layer of any operative material. Additionally, each wing portion 42 can comprise a composite material. For example, the wing portions 42 may include a spunbond fabric material, a bi-component spunbond material, a necked spunbond material, a neck-stretched-bonded-laminate (NBL) material, a meltblown fabric material, a bonded carded web, a thermal bonded carded web, a through-air bonded carded web or the like, as well as combinations thereof.

Each wing portion 42 can be joined to its corresponding side region of the napkin 20 in any operative manner. For example, the wing portion can be joined to the topsheet 26, the backsheet 28 or another napkin 20 component, as well as any combination thereof. The wing portion 42 can be attached with hotmelt adhesive, but any other operative adhesive or attachment mechanism may alternatively be employed.

In another feature, each wing portion 42, or any desired combination of the employed wing portion, can include a panel-fastener component 44 which is operatively joined to an appointed engagement surface of its associated wing. The panel-fastener can be configured to operatively attach to the wearer's undergarment and/or to any appointed, landing-zone portion of the napkin 20. For example, the panel-fastener 44 can include a system of interengaging mechanical fasteners, a system of adhesive fasteners, a system of cohesive fasteners or the like, as well as combinations thereof.

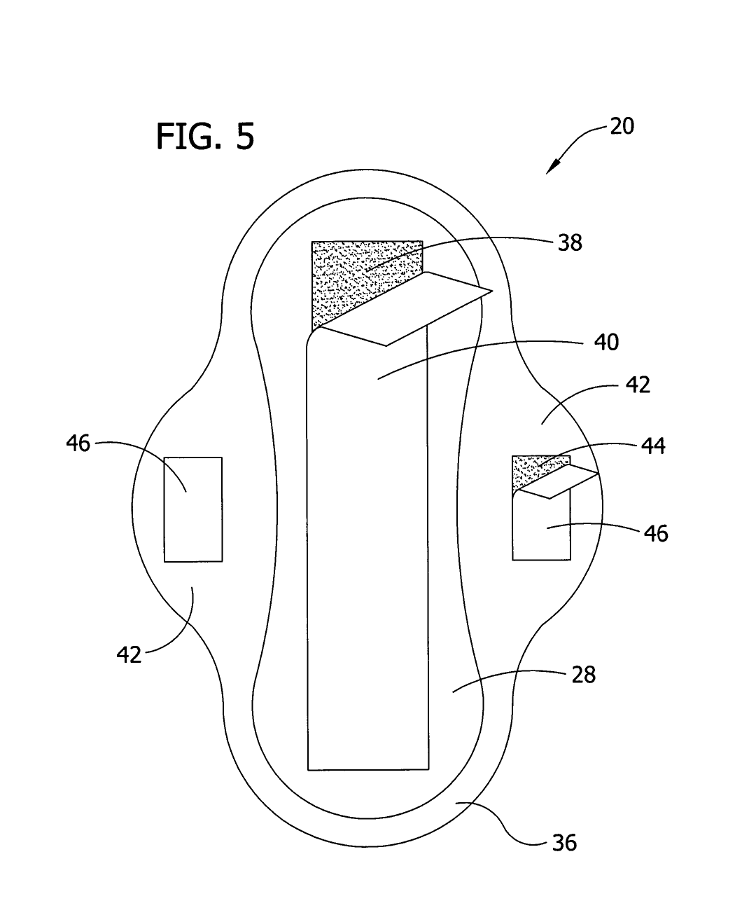

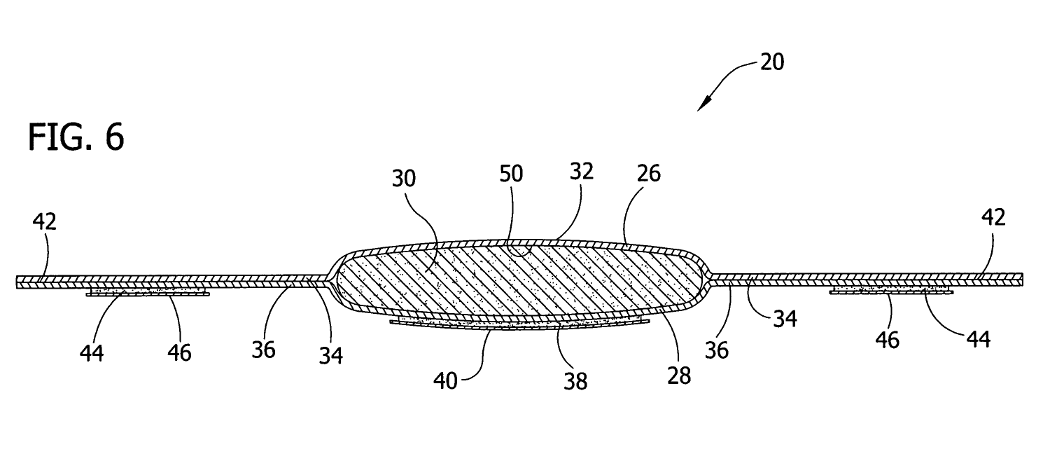

With reference to FIGS. 5 and 6, for example, either or both wing portions 42 can include a panel-fastener system 44 that alternatively incorporates an operative garment adhesive. The garment adhesive may be a solvent-base adhesive, a hotmelt adhesive, a pressure-sensitive adhesive, or the like, as well as combinations thereof. Each section of garment adhesive may be covered with a removable release material 46.

In the construction of the napkin 20, the various components (e.g., topsheet 26, backsheet 28, absorbent core 30, wing portions 42) may be assembled and held together with any operative securement mechanism or system. For example, the desired attachments or securements can include adhesive bonds, cohesive bonds, thermal bonds, ultrasonic bonds, pins, snaps, staples, rivets, stitches, welds, zippers, or the like, as well as combinations thereof.

The absorbent articles may optionally be folded and/or individually wrapped in various ways. For example, the absorbent articles may be folded and placed in an individual envelope or wrapper, or in other operative individual packaging. In a particular embodiment, the removable release material 40, 46 of each article can be configured to provide a wrapping layer for an individual article, and the wrapping layer can extend across at least two major surfaces of its corresponding absorbent article and can at least partially enclose the individual absorbent article. Accordingly, an individual absorbent article can have a removable release material which includes its corresponding, individual wrapper material.

In other embodiments, the wrapper may be formed from a separate piece of wrapper material using any methods known in the art. For example, the wrapper may be formed by sealing together various portions of a wrapper material in order to form an individual envelope or pouch that may encase the absorbent article. The wrapper generally comprises an interior and an exterior side, with the interior side facing the absorbent article and forming a hollow interior for receiving the absorbent article. The wrapper may sealed by heat-sealing, by crimp sealing, and/or by using a suitable adhesive, among other means. Such methods of sealing are commonly known in the art. In certain embodiments, the wrapper may optionally comprise opening aids, such as grasping means in the form of flaps, fins, tear strips, or the like, which increase ease of opening of the wrapper by improving accessibility of an end edge of the wrapper material. In this instance, the wrapper is generally opened by pulling on a free edge of the wrapper material, which breaks the sealing of the wrapper (e.g., breaks an adhesively sealed wrapper), thus allowing the absorbent article to be removed from the wrapper.

The wrapper may or may not be formed from a material that has sufficient transparency to allow an operative viewing of the article 20 by the user through the wrapping material. The wrapper can comprise any operative material. For example, the wrapping material can comprise a polymer film, a woven fabric, a nonwoven fabric, a composite laminate or the like, and combinations thereof.

The absorbent articles of the present disclosure may also be grouped together with several other absorbent articles to form a package of absorbent articles. The packages may comprise a plurality of the absorbent articles, preferably two or more individual absorbent articles, and more preferably comprise eight, ten, or more preferably twenty absorbent articles per package. The absorbent articles that are packaged together may be individually wrapped, as discussed above, or optionally may be packaged unwrapped. In one embodiment, the package of absorbent articles comprises a plurality of the same type of absorbent article (e.g., a package of napkins, a package of tampons, etc.). Alternately, the package of absorbent articles may comprise more than one type of absorbent article packaged together (e.g., a package comprising tampons, napkins, panty liners, and combinations thereof). It may also be desirable to package absorbent articles of different sizes and/or of different absorbent capacities together in a single package.

The package may comprise a soft or hard packaging material. Soft packaging includes flexible envelopes and packages made of sheet plastic and/or paper. Hard packaging includes generally less flexible packages made of plastic (e.g., tubs and buckets), metal or cardboard, and combinations thereof. Examples of suitable packaging materials include, for example, paper, cardboards, polymeric materials, coated paper, composite structures, or any other suitable packaging material. The package can have any operative configuration. For example, the package may comprise a bag, a carton, a container, a box, or the like, as well as combinations thereof. The various panels of the package can be flexible panels, rigid panels, semi-rigid panels, or the like, as well as combinations thereof. As discussed above with respect to wrappers, the package may also be formed by sealing together various portions of a packaging material (i.e., a packaging) in order to form a package encasing the absorbent articles. The packaging generally comprises an interior and an exterior side, with the interior side facing the absorbent articles and forming a hollow interior for receiving the absorbent articles. In certain embodiments, the package may comprise an opening mechanism, which provides access to the absorbent articles. Such opening mechanisms include, for example, a perforated section of packaging, a grasping means in the form of flaps, fins, tear strips, or the like, which increase ease of opening of the package by improving accessibility of an end edge of the package material, and combinations thereof.

In certain embodiments, the absorbent articles, wrappers, and/or packaging may comprise at least one food scent producing agent. As used herein, the term “food scent producing agent” is meant to refer to any compound or composition that produces a scent commonly associated with a particular type of food. Numerous examples of such food scent producing agents are known in the art and are readily available. For example, a food scent producing agent may produce a chocolate scent, a scent associated with a salty snack, or the scent released when an ice cream carton or wrapper is opened. Particularly preferred are scents associated with foods that are commonly craved by women during menstruation, such as chocolate scents and scents associated with salty snacks. Chocolate scents include, for example, scents associated with dark chocolate, milk chocolate, white chocolate, cocoa, hazelnut, mocha, and the like. Scents associated with salty snacks include, for example, the scent released when a bag of potato chips is opened, a popcorn scent, a pretzel scent, a tortilla chip scent, a cheese puff scent, a pizza scent, a fried food scent, a Chinese food scent, a nutty scent such as a cashew scent, an almond scent, a hazelnut scent, a peanut scent, a walnut scent, or other nutty scents, and the like.

Examples of compounds or compositions that produce a chocolate scent or a scent associated with a salty snack are known in the art. For example, compounds that produce a chocolate scent and/or taste include isoamyl cinnamate, anisyl alcohol, benzyl cinnamate, isobutyl hexanoate, isobutyl phenylacetate, benzyl ether, maltol, 2-methylbutyraldehyde, 2-ethyl-3,5-dimethylpyrazine, 2-ethyl-3,6-dimethylpyrazine, 3-ethyl-2-hydroxy-2-cyclopenten-1-one, 5-methyl-2-phenyl-2-hexenal, 4-methyl-2-phenyl-2-pentenal, 2,3,5,6-tetramethylpyrazine, 2,3,5-trimethylpyrazine, 2-methoxypyrazine, 4-methyl-5-vinylthiazole, 2,4,5-trimethylthiazole, butyl 2-methylbutyrate, and 3-methyl-1-pentanol, among others. Other chocolate scent producing agents include chocolate perfume oil, available from Escential Lotions & Oils, Inc. (Portland, Oreg.).

Compounds that produce a scent associated with a salty snack include, for example, compounds that produce an onion and/or garlic scent, compounds that produce a fatty butter scent, compounds that produce a fatty, oily scent, and compounds that produce a nutty scent, such as an almond scent, a hazelnut scent, a peanut scent, a walnut scent, or other nutty scents. Examples of onion and garlic scent producing agents include allyl disulfide, allyl mercaptan, allyl sulfide, butyl sulfide, garlic oil, 3-(methylthio)propionaldehyde, onion oil, furfuryl isopropyl sulfide, methyl propyl disulfide, propyl disulfide, o-toluenethiol, cyclopentanethiol, dimethyl trisulfide, ethyl thioacetate, methyl thiobutyrate, methyl 2-thiofuroate, allyl thiopropionate, furfuryl thiopropionate, methyl furfuryl disulfide, dicyclohexyl disulfide, 1-butanethiol, propyl mercaptan, 1,3-butanedithiol, 4-(methylthio)butanol, benzenethiol, isopropyl disulfide, 1,4-dithiane, methyl sulfoxide, 2-propanethiol, and cis/trans-3,4-dimethyl-2-mercapto-2,3-dihydrothiophene (DMMT), among others. Fatty, oily scent producing agents include, for example, allyl 2-ethylbutyrate, 2-methyl-1-butanol, amyl 2-furoate, butyl laurate, citral diethyl acetal, cuminaldehyde, diacetyl, 2-ethylbutyl acetate, ethyl decanoate, farnesol, furfuryl mercaptan, heptanol, 2,3-heptanedione, heptyl acetate, heptyl alcohol, 2,3-hexanedione, 6-methyl-5-hepten-2-one, myristic acid, octanoic acid, 2-octanol, octyl octanoate, propyl propionate, 2-ethyl-1-hexanol, trimethylamine, undecanoic acid, 2-acetylpyridine, 2-heptanol, α-angelicalactone, 2-pentanol, 3,5,5-trimethyl-1-hexanol, methyl 3-hydroxyhexanoate, parillaldehyde, 3-octanol, 3-methyl-2-butenol, isopropyl 2-methylbutyrate, methyl sulfoxide, 2-methyl-3-buten-2-ol, methyl stearate, and methyl decanoate. Nutty scent producing agents include almond scent producing agents such as acetophonone, benzylaldehyde, benzyl benzoate, benzyl ether, dihydrocoumarin, furfural, trans-2-hexenal, 5-methylfurfural, 3,4-hexanedione, 2-methoxy-3-methylpyrazine, 2,3-dimethylpyrazine, 2,4-dimethylbenzaldehyde, 2-methyl-3,5(or 6)-ethoxypyrazine, 6-amyl-α-pyrone, and 4-ethylbenzaldehyde, among others; hazelnut scent producing agents such as 2,3-diethylpyrazine, 3-ethyl-2-methylpyrazine, 2,4-heptadienal, 2-methoxy-3-methylpyrazine, 2,3,5-trimethylpyrazine, 2,4,5-trimethylthiazole, 2-acetyl-3,5(or 6)-dimethylpyrazine, 2-acetylthiazole, 2,3-diethyl-5-methylpyrazine, 2-methyl-3,5(or 6)-ethoxypyrazine, and methyl 2-methylpentanoate, among others; peanut scent producing agents such as 2-methoxy-4-vinylphenol and 2-methoxy-3-methylpyrazine, among others; walnut scent producing agents such as benzyl alcohol, 2-methylanisole, 2-phenylpropionaldehyde dimethyl acetal, 2,3-dimethylpyrazine, and 2-methoxyphenyl acetate, among others; and other nutty scent producing agents, such as acetanisole, benzaldehyde dimethyl acetal, methyl nonanoate, methyl valorate, phenylacetaldehyde, valeraldehyde, 2-acetylpyrazine, 2-ethyl-3,5(or 6)-dimethylpyrazine, 5-methylquinoxaline, 4-methyl-5-thiazoleethanol, 2-acetyl-3-ethylpyrazine, 2,5-dimethylpyrazine, 2,6-dimethylpyrazine, 4,5-dimethylthiazole, 2-ethylpyrazine, α-angelicalactone, 2-methoxypyrazine, 2-methylpyrazine, 4-methyl-5-vinylthiazole, 2-ethoxythiazole, pyrrole, 3-acetyl-2,5-dimethylfuran, 2-heptylfuran, 3-acetylpyridine, 2,6-dimethylpyridine, 2-isopropyl-4-methylthiazole, 3-octanol, 2-acetyl-5-methylfuran, thiazole, 2-ethyl-4-methylthiazole, 4-methylthiazole, 2-acetyl-3-methylpyrazine, and 2-methyl-3-propylpyrazine, among others. Fatty “butter” scent producing agents include, for example, acetanisole, acetoin, anisole, benzyl butyrate, butyl butyrllactate, butyl 10-undecenoate, isobutyric acid, δ-decalactone, diactyl, δ-dodecalactone, ethyl lactate, 2,3-heptanedione, 2,3-hexanedione, 2-methylheptanoic acid, 3-octanone, 2,3-pentanedione, 3,4-hexanedione, d-nonalactone, cis-3-hexenyl butyrate, trans, trans-2,4-undecadienal, 2-acetoxy-3-butanone, 1-penten-3-ol, 3-methyl-2-butenal, methyl sulfoxide, and 5-(hydroxyemthyl)furfural, among others. Other compounds that produce a scent associated with a salty snack include, for example, 4-hydroxy-2-(5)-ethyl-5(2)-methyl-3(2H)-furanone and furfuryl alcohol, among others.

Food scent producing agents are also commercially available. For example, chocolate scent producing agents may be purchased from Aldrich Chemical Co. (Milwaukee, Wis.), Arylessence, Inc. (Marietta, Ga.), Bell Flavors & Fragrances, Inc. (Northbrook, Ill.), Firmenich (Plainsboro, N.J.), Givaudan (Teaneck, N.J.), Shaw Mudge & Company (Shelton, Conn.), Symrise, Inc. (Teterboro, N.J.), and Eastman Chemical Co. (Kingsport, Tenn.). Salty snack scent producing agents may also be purchased commercially from these companies.

As described in more detail below, the food scent producing agent incorporated into and/or onto the absorbent articles, wrappers, and/or packaging may be encapsulated or entrapped within a thin layer of material. The food scent producing agent can be encapsulated in a number of shell-like materials including, for example, cellulose-based polymeric materials (i.e., ethyl cellulose), carbohydrate-based materials (e.g., cationic starches and sugars), polyglycolic acid, polylactic acid, and lactic acid-based aliphatic polyesters, and materials derived therefrom (e.g., dextrins and cyclodextrins). Other examples of suitable shell-like materials include liposomes, nanosomes, nanoparticles, collagen, gelatin, melamine resin, silicon resin, and combinations thereof. Alternatively, the encapsulating material may comprise a natural or synthetic polymer system (microsponge) such as, for example, acrylate polymers, acrylate copolymers, starch, silica, oat, and combinations thereof.

Encapsulating the food scent producing agent can protect the food scent producing agent from exposure to harsh environmental conditions which may result in premature degradation or release of the scent associated with the food scent producing agent. Encapsulation also allows for the separation of any incompatible components within the product, which allows greater flexibility in the components which can be used to make the product. Additionally, encapsulation allows for a controlled release of the food scent producing agent during use, handling, and/or storage of the absorbent article, wrapper, and/or packaging. A controlled release may include a triggered release, sustained release, targeted release, or a combination of these release mechanisms, wherein the food scent producing agent is released from the encapsulant by a number of mechanisms including, for example, pressure, ultraviolet light, capillary forces, and wetting with water, or is gradually released as the encapsulant breaks down over time. Other release mechanisms known in the art and suitable for use in accordance with the present disclosure include friction/pressure release, pH related release, water release, water evaporation release, and the like.

Typically, the particle size of the microencapsulated materials and the polymeric entrapment materials are from about 0.1 micrometers to about 40 micrometers, desirably from about 0.3 micrometers to about 20 micrometers, and still more desirably from about 0.5 micrometers to about 15 micrometers.

The microencapsulation shell thickness may vary depending upon the food scent producing agent utilized, and is generally manufactured to allow the encapsulated agent to be covered by a thin layer of encapsulation material, which may be a monolayer or thicker laminate layer, or may be a composite layer. The microencapsulation layer should be thick enough to resist cracking or breaking of the shell during handling or shipping of the product. The microencapsulation layer should also be constructed such that humidity from atmospheric conditions during storage, shipment, or wear will not cause a breakdown of the microencapsulation layer and result in a premature release of the food scent producing agent.

In one embodiment, the food scent producing agent is applied to and/or dispersed throughout all or a portion of the absorbent article. For example, the food scent producing agent may be applied to the topsheet, the backsheet, the removable release material, and/or may be dispersed throughout the absorbent core or other components of the absorbent article, as described above, so that the absorbent article itself has a food scent. Preferably the amount of food scent producing agent incorporated into the material (whether it be the topsheet, the backsheet, the removable release material, and/or the absorbent core or other components of the absorbent article) is about 0.01 to about 12 wt. % and preferably is about 0.05 to about 5 wt. % based on the total weight of the material. In certain embodiments, the absorbent articles may then be sealed within a wrapper and/or package, thereby containing the scent within the wrapper and/or package. Thus when the wrapper and/or package is opened, the food scent is released.

The food scent producing agent may be applied to the absorbent article using a variety of delivery systems for containing and delivering the food scent producing agent. The delivery system may be of any configuration including, for example, one that contains the food scent producing agent in a powder, particle, or flake form, or in a solution, a dispersion, a suspension, an emulsion or the like. It is generally preferable, although not required, that the food scent producing agent be encapsulated, as described above. The food scent producing agent may be impregnated into the absorbent article or applied to the absorbent article by any suitable means including, for example, spraying, printing, rolling, and pressing, among others.

In one embodiment, the food scent producing agent may be incorporated into a backsheet. Methods for incorporating scents into a backsheet are known in the art and disclosed in, for example, WO 2005/053761, incorporated herein by reference. Preferably, the amount of food scent producing agent incorporated into the backsheet is about 0.01 to about 12 wt. % and preferably is about 0.05 to about 5 wt. % based on the total weight of the backsheet.

In one embodiment, the food scent producing agent may be applied to all or a portion of the interior side of the wrapper and/or the packaging, and the wrapper and/or package is sealed. In this instance, the scent of the food scent producing agent may be emitted into the hollow interior of the wrapper and/or package. Since the hollow interior is generally confined until the package and/or wrapper is opened, the scent from food scent producing agent becomes concentrated in the hollow interior. Accordingly, the scent of the food scent producing agent is particularly strong in the hollow interior, prior to the package being opened, and may be smelled by a consumer when the individually wrapped absorbent article and/or package is opened. In certain embodiments, the scent may also permeate the absorbent article contained in the wrapper or package so that the absorbent article may have a food scent, even if the absorbent article does not actually comprise a food scent producing agent.

Alternately or in addition, the food scent producing agent may be applied to all or a portion of the exterior of the wrapper and/or the packaging. In this embodiment, the air immediately surrounding the exterior of the wrapper or packaging may contain the scent of the food scent producing agent, allowing a consumer to smell the food scent even without opening the package or wrapper.

As discussed above, the food scent producing agent may be encapsulated or non-encapsulated. If the food scent producing agent applied to the wrapper and/or packaging is non-encapsulated, it is generally preferred that the food scent producing agent be at least partially covered to prevent premature release of the scent and to preserve the freshness of the scent during shipping and storage. For example, the non-encapsulated food scent producing agent may be releasably covered with a removable release material, such as described above, a peel strip, a sticker, or other release materials so that when the release material is removed, the food scent is released. If the food scent producing agent applied to the wrapper and/or packaging is encapsulated, the food scent producing agent may be releasably covered, as described above, or optionally may remain uncovered. For example, in certain embodiments, the encapsulation may be designed so that the food scent producing agent is released under certain mechanisms, such as application of pressure or friction (e.g., scratch and sniff, handling by the consumer, etc.), exposure to ultraviolet light or other environmental stimuli, or may gradually be released as the encapsulant breaks down over time, as described above.

In one embodiment, the food scent producing agent may be incorporated into an adhesive present in and/or on the absorbent article, wrapper material, and/or packaging. As discussed above, various portions of the absorbent articles may be maintained in secured relation to other portions by bonding all or a portion of the adjacent surfaces to one another using, for example, various types of adhesives. For example, adhesives may be used to bond the topsheet to the absorbent core, the topsheet to the backsheet, the backsheet to the absorbent core, and/or to bond the topsheet, absorbent core, and backsheet together or to other components typically used to construct an absorbent article, such as a wing portion, or other components that are well known to one skilled in the art. Additionally, the absorbent articles may comprise positioning adhesives, such as a garment adhesive 38 that may be used to attach the absorbent article to an undergarment, or a panel-fastener component 44 that may be used to secure the wings to an undergarment. Adhesives may likewise be used to bind portions of the wrapper material and/or packaging together to form a wrapper for an individual absorbent article or a package, respectively, or to releasably seal the wrapper or package closed, as discussed above.

The type of adhesive used in connection with the absorbent articles, wrapper and/or packaging described herein is not limited and may be any adhesive known in the art and suitable for use in the manufacture of absorbent articles. Suitable adhesive compositions include, for example, pressure-sensitive adhesives, hot melt adhesives, solvent-base adhesives or the like, as well as combinations thereof.

In certain embodiments, some or all of the adhesives present in and/or on the absorbent product, wrapper, and/or packaging have incorporated therein a food scent producing agent. Methods for incorporating scents into adhesive material are known in the art. For example, in one embodiment, the food scent producing agent may be incorporated into a hot melt adhesive used to bind together various parts of the absorbent article, wrapper, and/or package, as discussed above. Methods for incorporating scents into hot melt adhesives are known in the art and disclosed in, for example, U.S. Patent Application No. 2003/0105183, incorporated herein by reference, which describes hot melt adhesives that comprise non-encapsulated fragrance compositions. Briefly, applying this method to the present disclosure, food scent producing agents that have a closed cup flash point greater than about 100° F., preferably greater than about 150° F., and more preferably greater than about 200° F., as determined by ASTM method D93-00 (Flash Point by Pensky-Martens Closed Cup Tester) may be added, with stirring, at any time during the preparation of the hot melt adhesive. If a food scent producing agent has a flash point less than about 100° F., it may be modified by, for example, modifying the amounts and type of raw materials used to produce the food scent or by adding other ingredients, so as to increase or otherwise raise the flash point to the required threshold. Various methods of modifying the food scent producing agents are within the skill of the art and include, for example, substituting, functionalizing, derivitizing, and/or forming adducts of the raw materials used to produce the food scent.

Methods and materials used to produce hot melt adhesives are well known in the art. Any base polymer suitable for use in formulating a hot melt adhesive may be used including, for example, amorphous polyolefins, ethylene-containing polymers and rubbery block copolymers, as well as blends thereof. Hot melt adhesive compositions based on ethylene/vinyl acetate copolymers, isotactic or atactic polypropylene, styrene-butadiene, styrene-isoprene, or styrene-ethylene-butylene A-B-A or A-B-A-B block copolymers or mixtures thereof may be used. In addition to the base polymer, the hot melt adhesive compositions may also contain tackifiers, oils and/or waxes as well as conventional additives including stabilizers, anti-oxidants, pigments and the like. The hot melt adhesive compositions are typically prepared by blending the components in the melt at a temperature of about 100° C. to 200° C. until a homogenous blend is obtained, typically about 2 hours. Methods of blending are known in the art.

Typically, the food scent producing agent is added to the hot melt adhesive in an amount effective to be noticable, and remain noticable to the user following cure and to the user of the absorbent article manufactured using the scented adhesive. As will be appreciated by one skilled in the art, the amount of food scent producing agent incorporated into the adhesive will depend on the origin, composition, purity, type of modification/derivation, degree and type of adulteration and the like of the food scent producing agent. Certain food scent producing agents may be useful in amounts of from about 0.001 to about 1.0 part per 100 parts of the adhesive composition. Typically, the food scent producing agent used in the adhesive will be from about 0.05 to about 15 weight percent, and more preferably between about 2 and about 12 weight percent of the adhesive composition.

In other embodiments, adhesive comprising a food scent producing agent may be used in combination with a removable release material, such as a peel strip or a tear strip. For example, a food scent producing agent (encapsulated or non-encapsulated) may be incorporated into an adhesive, such as described herein, and covered with a removable release material. In this instance, the removable release material may be pulled from the adhesive, thereby uncovering the adhesive comprising the food scent producing agent. By removing the removable release material from the adhesive, the food scent associated with the food scent producing agent is also released. In one embodiment, an encapsulated food scent producing agent is embedded in the adhesive and a removable release material is applied on top of the adhesive. When the removable release material is removed from the adhesive and peeled back, the sheer force across the top of the encapsulated food scent producing agent causes it to break and release the food scent.

In one particular embodiment, the food scent producing agent is embedded in the garment adhesive 38 and/or in an adhesive used to fasten the wings 44 using a method such as those known in the art and described herein, and the adhesive is covered with a removable release material 40, 46, such that when the removable release material 40, 46 is peeled back from the adhesive, the food scent is released.

In another embodiment, an adhesive comprising a food scent producing agent (encapsulated or non-encapsulated) is applied to the packaging material and/or wrapper and at least a portion of the adhesive is covered with a removable release material, such that when the removable release material is peeled back from the adhesive, the food scent is released.

In yet another embodiment, an adhesive comprising a food scent producing agent (encapsulated or non-encapsulated) is used to seal the wrapper and/or package closed, such that when the wrapper and/or package is opened, e.g., by pulling on a flap or fin or tear strip, as described above, the adhesive seal is broken and the food scent is released.

In another embodiment, the food scent producing agent may be incorporated into the wrapper material that encloses an individual absorbent article and/or may be incorporated into the packaging material, backsheet, removable release material, or any other component of the absorbent article that may comprise polymeric material. Methods for incorporating scents into plastic polymeric compositions are known in the art and disclosed in, for example, WO 88/01503, incorporated herein by reference. Briefly, food scent producing agents may be combined with low to medium molecular weight functional polymers to produce a liquid additive concentrate or compound. This liquid additive concentrate is combined with a functional polymeric chemical compound (“polymer P1”) that has a high solubility in the liquid additive (i.e., approaches forming a single phase with the liquid additive such that the physical characteristics of the compounds are difficult to separate). The resulting mixture may be combined with the base polymer used to form the wrapper material, packaging material, back sheet, removable release material, etc. to form a scented polymeric material.

The molecular weight of polymer P1 is preferably in the range of 500 to 2500. Higher molecular weight polymers that are highly polar may also be used. Polymer P1 is preferably selected from the group consisting of ethylene acrylic acid copolymer, ethylene vinyl acetate copolymer, ionomer, polyamide, ester wax, amide wax, montan wax, maleic anhydride grafted polyethylene, polyethylene glycol, hydroxystearamide wax, hydroxywax, carnuba wax, alphamethylstyrene, and more preferably is selected from the group consisting of ethylene acrylic-acid copolymers, ethylene methacrylic acid copolymers, ethylene vinyl acetate copolymers, low molecular weight ionomers, polyamides, ester waxes (oxidated low molecular weight polyethylene polymers), and amide waxes. Mixtures of two or more of these compounds are also effective.

The choice of polymer P1 needed to obtain maximum solubility and physical characteristics will vary depending on the composition of the food scent producing agent in the liquid additive concentrate. It is usually necessary to determine the solubility of polymer P1 by trial and error. For example, the solubility characteristics of the mixture of polymer P1 and the liquid additive may be determined by stirring polymer P1 and the liquid additive in a heated flask sufficient to melt the polymer and the liquid additive. After mixing for about five minutes, the mixture is allowed to cool in a slab on aluminum foil in a plastic bag. The mass is observed over a period of days at ambient conditions to determine any spew, bloom, or migration of the liquid out of the mass to the surface. Other physical characteristcs of the mix, such as hardness and hot strength, may also be observed. In addition, the quality of the scent given off by the mass may be compared to the original scent prior to processing to determine if polymer P1 is suitable for use with the liquid additive.

Once a suitable polymer P1 is chosen, the viscosity and flash point of the liquid additive/polymer P1 mixture may be increased to a point where release of the fragrance is highly controlled. This increase in viscosity and flash point by the inclusion of P1 polymer helps retain the bulk of the scent in the liquid additive/polymer P1 composition when this composition is processed into the base polymer used to form, for example, a wrapper material, packaging material, back sheet, removable release material, etc. that has a food scent.

Other methods for incorporating fragrances into packaging material is described in U.S. Patent Application No. 2004/0028779, herein incorporated by reference. Briefly, applying this method to the present disclosure, a food scent producing agent may be incorporated into the wrapper material, packaging material, back sheet, removable release material, and/or any other component of the absorbent article that may comprise in part or in whole a plastic material. For example, a food scent producing agent may be mixed with the plastic compound used to produce the wrapper material, packaging material, etc. so that the food scent producing agent saturates the plastic compound at the molding temperature of the plastic compound and supersaturates the plastic compound at ambient temperature. In this manner, the food scent producing agent will fully mix with the plastic compound prior to the plastic compound being molded or extruded into the packaging material, wrapper material, etc. and, as this material begins to cool, the food scent producing agent will begin to precipitate out of solution. Since the plastic compound is a solid, the supersaturation of the food scent producing agent will cause the food scent producing agent to very gradually bloom to the exposed surfaces of the packaging material, wrapper material, etc. The concept of “bloom” is well known in the plastics industry and refers to the migration of a liquid or a solid material from the composition of the plastic to the surface of the plastic. In the present case, the bloom of the supersaturated food scent producing agent will continue over time until the concentration of the food scent producing agent within the plastic compound falls to a concentration corresponding to the saturation point of the plastic compound, and ceases when the concentration of the food scent producing agent in the plastic compound becomes equal to its solubility at a given temperature. Thus, the scent may remain for as long as the food scent producing agent blooms from the plastic, which can be several months or years, or until the package and/or wrapper is opened.

In one embodiment, a wrapper for an individual absorbent article and/or a package may be manufactured using the above described method. In this instance, the food scent producing agent may bloom out to both the interior and exterior surface of the package and/or wrapper. Accordingly, both the interior and exterior of the package and/or wrapper continues to be scented over time. Since the packaging material or wrapper material may be formed either entirely from the mixture of plastic, or the outer and inner layers of a packaging material or wrapper material may be formed from the mixture, the scent of the plastic may be emitted into the interior of the package. As such, the hollow interior of the package and/or wrapper may contain the scent of the food scent producing agent that was added to the plastic. Since the hollow interior is confined until the package and/or wrapper is opened, the food scent producing agent exiting the plastic becomes concentrated in the hollow interior. Accordingly, the scent of the food scent producing agent added to the plastic is particularly strong in the hollow interior, prior to the package being opened, and may be smelled by a consumer when the individually wrapped absorbent article and/or package is opened. In addition, for packaging material, wrapper material, or other plastic material that is molded from a plastic mixed with a food scent producing agent, the air immediately surrounding the exterior of the material may contain the scent of the food scent producing agent. Since the exterior of the package and/or wrapper also emits the scent of the food scent producing agent, a consumer may be able to smell the food scent even without opening the package.

Other methods for incorporating scents into packaging are known in the art and described in, for example, EP 1 384 682 and WO 2004/034819, herein incorporated by reference. Briefly, in the method described in WO 2004/034819, food scent producing agents are entrapped, encapsulated, impregnated, or embedded in a suitable solid or semi-solid matrix that may be attached to the exterior or interior of a package. The scent is slowly released from the matrix into the atmosphere in a controlled manner for prolonged periods of time.

Applying this method to the present disclosure, a food scent producing agent may be incorporated into a solid or semi-solid matrix, and the resulting scented matrix attached to a portion of the absorbent article, wrapper, and/or package to provide a slow release of the desired food scent. For example, the scented matrix may be obtained by combining a food scent producing agent with UV crosslinkable monomers and oligomers and suitable photo-initiators, and solidifying the mixture by exposing it to UV light so that the food scent producing agent is trapped within a solidified matrix. The scented matrix may also be obtained by combining the food scent producing agent with heat cross-linkable monomers and oligomers, which become solid upon heating in the presence of thermal initiators. In other embodiments, the matrix may be prepared using wax. In this instance, wax may be melted, after which the food scent producing agents are added, and the resulting mixture mixed until a solution or emulsion is formed while the wax is in its liquid state, above its melting point. This scented wax mixture may then be poured into a mold to form a specific shape which may subsequently be attached to the interior and/or exterior surface of the package and/or wrapper, for example, by gluing, sticking, heat attaching or the like. Alternatively, the scented wax mixture may be directly applied to the package and/or wrapper, for example in a specially constructed recess and left to harden onto an interior wall of the package and/or wrapper. Upon cooling, the wax immediately becomes a solid patch or piece that contains the food scent producing agent.

The ability of the matrix to deliver a strong scent impact depends on various preparation parameters and conditions that can be controlled including, for example, the food scent producing agent/matrix ratio, the type of the matrix used (which determines the diffusion of the food scent out of the matrix), the degree of crystallinity and/or cross-linking of the matrix (where applicable), the ratio between surface area and volume of the matrix, the overall dimensions, the volatility of the food scent producing agent, etc. All the above parameters can be chosen so that the release of the food scent from the matrix is slow and controlled.

When it is intended that the matrix slowly release the food scent, typically most of the scented matrix is composed of the matrix itself, and not of the food scent producing agent. In this instance, the food scent producing agent typically comprises no more than 25% w/w of the scented matrix, preferably no more than 20%, more preferably no more than 15%, most preferably no more than 10% w/w of the scented matrix, so that the effect is of slow controlled release. It will be appreciated by one skilled in the art that these percentages may vary if a one time strong scent release is desired.

In certain embodiments, the scented matrix may be enclosed with a permeable membrane to prevent the matrix from contacting the absorbent articles within the package and/or wrapper, but that allows the food scent associated with the food scent producing agent to enter the surrounding air, such as the hollow interior of the package or wrapper or the air immediately surrounding the exterior of the scented matrix. Optionally, the scented matrix may be covered by an outer, impermeable layer, such as a removable relase material (e.g., peel strip, sticker, tear seal, etc.), discussed above, that when removed, allows access to the scented matrix.

In other aspects, the absorbent articles, wrappers, and/or packages may comprise a food related reward. The absorbent articles, wrappers, and/or packages may comprise the food related reward as an alternative to, or in addition to, a food scent producing agent. In one embodiment, the absorbent article, wrapper, and/or package may comprise a food related reward and a food scent producing agent that produces a food scent associated with the food related reward.

As used herein, the term “food related reward” is meant to refer to a food item, food scented item, food benefit coupon, or other food related product or service, and combinations thereof. Particularly preferred food related rewards are those associated with foods that are commonly craved by women during menstruation, such as chocolate or chocolate flavored foods, and salty foods. Examples of such foods include chocolate related foods including a piece of chocolate, chocolate candy bars, chocolate flavored candy, chocolate cookies, chocolate cakes and pies, chocolate ice cream, and mocha (i.e., chocolate-coffee) flavored treats, and salty foods, such as potato chips, pretzels, and related snacks, crackers, popcorn, french fries, nuts, pizza, and the like.

The food scented item may be any item or product having a food scent, but particularly preferred are those items that may be used to pamper or provide comfort to a woman during menstruation. For example, food scented items may include food scented spa and bath products such as food scented bath soaps, oils, and gels, food scented face and body creams and lotions, food scented candles, food scented absorbent articles and packages, as described herein, and combinations thereof. Particularly preferred are items that have a chocolate scent or a scent associated with a salty snack.

The food related reward may also be a food benefit coupon. The term “food benefit coupon” is meant to include any coupon, certificate, or the like that may be redeemed for a food related reward, such as a food item, a food scented item, or other food related products or services, or that may be used to obtain a redeemable coupon or certificate. For example, the food benefit coupon can be a coupon that is redeemable for a predetermined monetary amount as applied to a particular food related item, such as a food item, food scented item, or other food related products or service, as described herein. The food benefit coupon may also be a coupon which is redeemable for a free food related item. In one embodiment, the food benefit coupon may comprise an authentication code. Any operative authentication code may be employed. For example, the authentication code can include a bar code, a number code, a text code, an alpha-numeric code or the like, as well as combinations thereof. In one aspect, the authentication code can be machine-readable. The authentication code on the food benefit coupon may be used to acquire a food related item or a discount on a food related item, or may be used to acquire a certificate or coupon which is redeemable for a food related item, as described in more detail herein.

The absorbent articles, wrappers, and/or packages of the present disclosure may comprise one or more food related rewards. For example, the packages and/or individually wrapped absorbent articles described herein may comprise therein a food related item, such as a food item, a food scented item, and combinations thereof. In one particular embodiment, a food item such as one or more pieces of chocolate or a chocolate bar, may be packaged with a plurality of absorbent articles and contained in the hollow interior of the packaging and/or may be wrapped with an individual absorbent article and contained in the hollow interior of the wrapper.

In a preferred embodiment, the absorbent articles, wrappers, and/or packages of the present disclosure comprise at least one food benefit coupon. The food benefit coupon may be packaged with a plurality of absorbent articles and contained in the hollow interior of the packaging and/or may be wrapped with an individual absorbent article and contained in the hollow interior of the wrapper.

Alternately or in addition, the food benefit coupon may be on a removable release material, such as those described herein. For example, the food benefit coupon may be located on an exposed surface of a removable release material, which is arranged to face outwardly toward the consumer. Alternately, the food benefit coupon may be positioned on an interior surface of the removable release material, and the removable release material may or may not be configured to be sufficiently transparent to allow an operative viewing of the food benefit coupon through the removable release material.

In one embodiment, the food benefit coupon may be positioned on a removable release material 40, 46 used to cover the garment adhesive 38 or the wing panel fasteners 44. Optionally, the food benefit coupon can be located on a surface of the removable release material in a manner that allows the food benefit coupon to be at least partially visible to the user when the removable release material is in its storage position and the fresh, unused article is presented to the user when the package or wrapper is opened.

The food benefit coupon may also be releasably attached to the wrapping and/or the packaging. For example, a removable release material, such as a removable label, peel strip, tear strip, or other removable layer, may be attached to the wrapper and/or the package, as described herein. The food benefit coupon may be located on an exposed surface of the removable release material, which is arranged to face outwardly toward the consumer, or may be positioned on an interior surface of the removable release material.

The food benefit coupon may be applied to the removable release material by employing any operative technique (e.g., embossing) without departing from the scope of the present disclosure. For example, the food benefit coupon can be printed on the removable release material using conventional printing techniques.

The food benefit coupon can include a predetermined set of text printed on the removable release material. Alternatively, the food benefit coupon can include a distributed pattern of perforations, a distributed pattern of embossments or the like, as well as combinations thereof.

In another aspect, the food benefit coupon can be configured to be substantially, non-recognizable to ordinary human vision when the removable release material is in its storage position, and is configured to become operatively recognizable to ordinary human vision upon a removal of the removable release material from the article, wrapper, and/or package. The change in the ability to recognize the food benefit coupon can be provided by any operative structure or technique. For example, the food benefit coupon can be configured to become operatively recognizable to ordinary human vision upon a removal of the removable release material 40, 46 from the garment adhesive 38 or panel fastener adhesive 44. In a particular configuration, the food benefit coupon can be located on a body side surface of the removable release material.

As another example, the change in the ability to recognize the food benefit coupon can include a code or text provided by an array of perforations where perforations and the associated code or text becomes more readily discernable after the food benefit coupon has been removed from the article. In a further example, the food benefit coupon can include a code or text provided by a color-shifting ink or an ink that becomes activated and visible to human sight when the ink has been exposed to selected wavelengths of light. Still another example of such change in the recognition ability can include a code or text which has been provided by a pattern of ink, adhesive or other coating, and which subsequently becomes revealed after the food benefit coupon has been removed from the article.

The present disclosure also provides methods for rewarding consumers for the purchase of absorbent products, such as the absorbent articles and packages of absorbent articles described herein. For example, in one embodiment, the method comprises determining when a consumer purchases an absorbent product, and authorizing a third party to distribute a food related reward to the consumer. As used herein, the term “consumer” is meant to include both the user of the absorbent product, as well as a purchaser of the absorbent product.

A variety of means may be used to determine when an absorbent product has been purchased. In one embodiment, the absorbent product may comprise a unique identifier, such as an individual bar code, a RFID tag, a product code, a number code, a text code, an alpha-numeric code or the like. The unique identifier may be located on a food benefit coupon attached to or packaged with the absorbent product or may be found on the absorbent product or package itself. In certain instances, the unique identifier may be used to determine when the absorbent product has been purchased. This may be done automatically, such as by reading or scanning the bar code or RFID tag associated with the product, or may be done by means of interactive communication with the consumer. Examples of such interactive communication may include instances where the consumer inputs information about the product into a user interface (e.g., speaking the product identity, selecting the product type from a menu of items in a graphical user interface, typing in a code or product name, pressing a button specific to a product category, etc.). In other embodiments, the determination of product purchase may be made by visual means (e.g., a salesperson in a store sees the purchased product or a receipt for the purchased product).

Once it has been determined that a purchase of an absorbent product has been made (or is about to be made), a third party is authorized to distribute a food related reward to the consumer. The third party may include, for example, a coupon dispensing machine, a salesperson, a delivery system (e.g., the United States Postal Service, FedEx, UPS, etc.), a product dispensing machine, and combinations thereof. In certain embodiments, a communication may be sent to the third party, indicating that the absorbent article has been purchased by the consumer, and authorizing the third party to distribute a food related reward to the consumer. Any suitable communication may be used, such as electronic signals, visual signals, auditory signals, magnetic signals, and combinations thereof.

The food related rewards may be distributed in a variety of ways. In one particular embodiment, the consumer may purchase the absorbent product in a store and, upon determining that an absorbent product has been purchased, a salesperson may offer the consumer a food related reward. Alternately, if the food related reward is a food benefit coupon, a coupon dispenser with a built-in printer may provide the coupon to the customer at the time of purchase. In certain instances, a communication may be sent to the third party to signal the third party that an absorbent article has been purchased and that the third party is authorized to distribute a food related reward to the consumer. For example, a visual signal, such as an image or textual reminder, or an auditory signal, such as a beep or noise, may be sent to the salesperson to remind him or her that a food related reward should be distributed. In another example, an electronic signal may be sent to the coupon dispenser to signal that a food benefit coupon should be printed.

The food related reward may also be present as part of a display located in the general proximity of the absorbent products. In this embodiment, the consumer may select the desired absorbent product to be purchased and also take a food related reward from the display. The consumer is then allowed to keep the food related reward upon purchase of the absorbent product.

In another example, the food related reward may be distributed from a product dispensing machine. In this embodiment, when the consumer purchases the absorbent product from the machine, the machine may be triggered to dispense a food related reward to the consumer.

In one particular embodiment, a consumer may input into a user interface information about the purchased product, as described herein. Based on this information, a third party is authorized to distribute a food related reward to the consumer. The means for distributing the food related reward may vary depending on the particular type of food related reward. For example, if the food related reward is a food benefit coupon, the coupon may be distributed to the consumer by means of a coupon dispenser or other printing device in connection with the interface, or may be delivered to the consumer by means of a delivery system, such as through the mail. If the food related reward is a food item or a food scented item or other food related product, the reward is typically delivered to the consumer by means of a delivery system such as the United States Postal Service, FedEx, UPS, etc.

In another aspect, the consumer may be provided with a choice of food related rewards. According to this method, it is determined when the consumer has purchased an absorbent product, as described above. The consumer is provided with a plurality of choices of food related rewards from which to select. Once the consumer selects at least one of the rewards from the plurality of choices, the selected food related reward (or rewards) is distributed to the consumer.

The food related reward may be selected by the consumer in a wide variety of ways. In one embodiment, the consumer may select a food related reward from a display or collection of rewards. For example, the display or collection of rewards may be available at the point of sale, such as at a check out location in a store. In one particular embodiment, the consumer may purchase the absorbent product in a store and, upon purchase of the product, a salesperson may offer the consumer a choice of several different food related rewards from which to select. The consumer may then select at least one of the offered food related rewards, and receive the reward from the salesperson. Alternately, the food related rewards may be present as part of a display located in the general proximity of the absorbent products. In this embodiment, the consumer may select the desired absorbent product to be purchased and also make a selection from the display of food related rewards. The consumer is then allowed to keep the food related reward upon purchase of the absorbent product.

In another example, the display or collection of food related rewards may be contained within a product dispensing machine. In this embodiment, the consumer purchases the absorbent product from the machine, and may be prompted to select at least one food related reward from a plurality of choices offered by the machine. After making a selection, the dispensing machine distributes the selected reward to the consumer.

The food related reward may also be selected electronically, such as through an electronic display or website. For example, the purchased product may be automatically identified through the product's unique identifier, such as by reading a bar code or RFID tag associated with the product or other automatic identification system, or wherein the user can provide input to identify the product (e.g., speaking the product identity, selecting the product type from a menu of items in a graphical user interface, typing in a code or product name, pressing a button specific to a product category, etc.), such as through a user interface. A user interface may include, for example, a mobile, cellular telephone; an electronic, personal data assistant; an electronic computer configured with an operative user-interface, such as a graphical user interface; a website; an electronic display; or the like. Once the purchased product has been identified, a plurality of choices of food related rewards is displayed on the interface for selection by the consumer. The consumer may then input the reward selection into the interface.

The means for distributing the food related reward may vary depending on the food related reward that is selected. For example, if the selected food related reward is a food benefit coupon, the coupon may be distributed to the consumer by means of a coupon dispenser or other printing device in connection with the interface, or may be delivered to the consumer by means of a delivery system, such as through the mail. If the selected food related reward is a food item or a food scented item or other food related product, the reward is typically delivered to the consumer by means of a delivery system such as the United States Postal Service, FedEx, UPS, etc.

In one particular embodiment, a certain point value may be assigned to each type of product purchased and, based on a consumer's accumulated point total, the choice of food related rewards may change. For example, the consumer may be identified by reading a loyalty card that is swiped, reading an RFID tag or bar code on the loyalty card, identifying a personal code or password that may be entered into the user-interface by the consumer, or by any other known means to identify the consumer. Once the consumer has been identified, a consumer database containing information on the consumer may be accessed. For example, the amount of total points accumulated by the consumer may be accessed from the consumer database. The newly purchased product may be identified and a point value assigned to that product based on the product's unique identifier, as described above. The point value of the newly purchased product may be added to the consumer's previous point value to determine the new accumulated point total for that consumer. Food related rewards that are available for selection at the consumer's point level may be displayed for the consumer. The consumer may opt to select one or more of the food related rewards displayed, or alternately may choose to continue accumulating points.

The present disclosure also provides a system for distributing a reward to a consumer. The system may comprise means for monitoring consumer purchases to determine when the consumer purchases an absorbent product. Examples of such means include unique product identifiers such as those described above, consumer input of product information into a user interface, as described above, visual means such as a salesperson observing the purchase or seeing a receipt for the absorbent product, and the like. The system may further comprise means for signaling a third party when the absorbent product is purchased by the consumer, such as electronic, visual, auditory, and/or magnetic signals, and means for distributing a food related reward to the consumer in response to the purchase of the absorbent product by the consumer, such as the various distribution methods described above.

In another embodiment, a system for distributing a reward to a consumer is provided. The system may comprise means for monitoring consumer purchases of an absorbent product. The system may also comprise means for maintaining a consumer database containing information on monitored purchases by the consumer. The information contained in the consumer database may include previous purchases by the consumer, accumulated point values for previous purchases by the consumer, as discussed above, and other similar information. Preferably, the system further comprises means for receiving from the consumer a selection of a food related reward from a plurality of choices (e.g., a user interface and other such means, as discussed above). The system may further comprise means for distributing the selection of the food related reward to the consumer as a function of the information in the consumer database, such as the various distribution methods described above.

Having described the disclosure in detail, it will be apparent that modifications and variations are possible without departing from the scope of the disclosure defined in the appended claims.

When introducing elements of the present disclosure or the preferred embodiments(s) thereof, the articles “aa”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the disclosure are achieved and other advantageous results attained.

As various changes could be made in the above products and methods without departing from the scope of the disclosure, it is intended that all matter contained in the above description and shown in the accompanying drawings shall be interpreted as illustrative and not in a limiting sense.

What is claimed is:

1. A package of absorbent articles comprising:
a plurality of individual absorbent articles;
a packaging comprising a pocket having a hollow interior for receiving said plurality of absorbent articles and an opening mechanism, which provides access to said absorbent articles; and
a food scent producing agent;
wherein the food scent produced by the food scent producing agent is selected from the group consisting of a chocolate scent and a scent associated with a salty snack.

2. The package of claim 1 wherein the food scent producing agent is integrated into the packaging.

3. The package of claim 1 wherein the food scent producing agent is disposed on the packaging.

4. The package of claim 3 wherein the food scent producing agent is disposed on the exterior of the packaging.

5. The package of claim 3 wherein the food scent producing agent is disposed on the interior of the packaging.

6. The package of claim 3 wherein the packaging further comprises a removable release material releasably secured to the packaging over at least a portion of the food scent producing agent.

7. The package of claim 6 wherein the removable release material comprises a coupon for a food related item.

8. The package of claim 1 wherein the packaging releases the food scent when opened.

9. The package of claim 1 wherein the absorbent articles are individually wrapped in a wrapper.

10. The package of claim 9 wherein the food scent producing agent is disposed on the wrapper.

11. The package of claim 9 wherein the food scent is released when the wrapper is opened.

12. The package of claim 3 wherein a scented matrix is disposed on the packaging, the scented matrix comprising no more than about 10% w/w of the food scent producing agent.

13. The package of claim 1 wherein the food scent is a chocolate scent.

14. The package of claim 1 wherein the absorbent articles are selected from the group consisting of diapers, training pants, tampons, napkins, panty liners, interlabial pads, and combinations thereof.

15. The package of claim 1 further comprising a food related reward.

16. The package of claim 1 wherein the food scent producing agent is encapsulated.

17. A package of absorbent articles comprising:
a plurality of absorbent articles;
a packaging comprising a pocket having a hollow interior for receiving said plurality of absorbent articles and an opening mechanism, which provides access to said absorbent articles; and
a food benefit coupon.

18. The package of claim 17 wherein the food benefit coupon is selected from the group consisting of a coupon for a food item, coupon for a food scented item, and combinations thereof.

19. The package of claim 17 wherein the food benefit coupon is releasably attached to the exterior of the packaging.

20. The package of claim 17 wherein the food benefit coupon is within the hollow interior.

21. The package of claim 17 wherein the absorbent articles are individually wrapped in a wrapper.

22. The package of claim 21 wherein the food benefit coupon is within the wrapper.

23. The package of claim 17 wherein the food is selected from the group consisting of chocolate, salty foods, and combinations thereof.

24. The package of claim 17 further comprising a food related reward selected from the group consisting of a food item, a food scented item, and combinations thereof.

25. The package of claim 24 wherein the food related reward is selected from the group consisting of chocolate, a chocolate scented item, and combinations thereof.

26. The package of claim 17 wherein the absorbent articles are selected from the group consisting of diapers, training pants, tampons, napkins, panty liners, interlabial pads, and combinations thereof.

27. An absorbent article comprising:
an absorbent structure; and
a food scent producing agent;
wherein the food scent produced by the food scent producing agent is selected from the group consisting of a chocolate scent and a scent associated with a salty snack.

28. The absorbent article of claim 27 further comprising an adhesive.

29. The absorbent article of claim 28 wherein the food scent producing agent is disposed within the adhesive.

30. The absorbent article of claim 29 further comprising:
a topsheet having a bodyside surface oriented for facing a wearer when the absorbent article is worn and a backside surface opposite the body facing surface; and
a backsheet having an absorbent facing surface and an outer surface;
wherein the absorbent structure is positioned between the topsheet and the backsheet.

31. The absorbent article of claim 30 wherein the adhesive is disposed on the outer surface of the backsheet.

32. The absorbent article of claim 31 further comprising a removable release material releasably secured to the adhesive, which when removed, allows for release of the food scent.

33. The absorbent article of claim 32 wherein the removable release material comprises a food benefit coupon.

34. The absorbent article of claim 29 wherein the adhesive comprises from about 2 wt. % to about 12 wt. % of the food scent producing agent.

35. The absorbent article of claim 27 wherein the food scent producing agent is encapsulated.

36. The absorbent article of claim 27 wherein the food scent is a chocolate scent.

37. The absorbent article of claim 27 wherein the absorbent article is selected from the group consisting of diapers, training pants, tampons, napkins, panty liners, interlabial pads, and combinations thereof.

38. A method for rewarding a consumer for a purchase, the method comprising:
determining when the consumer purchases an absorbent product; and
authorizing a third party to distribute a food related reward to the consumer when the determining indicates that the absorbent product has been purchased by the consumer.

39. The method of claim 38 wherein the authorizing comprises sending a communication to the third party indicating that it has been determined that the absorbent product has been purchased by the consumer.

40. The method of claim 39 further comprising distributing by the third party the food related reward to the consumer in response to the communication.

41. The method of claim 38 wherein the absorbent product is selected from the group consisting of diapers, training pants, tampons, napkins, panty liners, interlabial pads, and combinations thereof.

42. The method of claim 38 wherein the third party is selected from the group consisting of a coupon-dispensing machine, a salesperson, a delivery system, a product dispensing machine, and combinations thereof.

43. The method of claim 38 wherein the food related reward is selected from the group consisting of a food benefit coupon, a food item, a food scented item, and combinations thereof.

44. The method of claim 38 wherein the food is selected from the group consisting of chocolate, salty foods, and combinations thereof.

45. The method of claim 39 wherein the communication is selected from the group consisting of electronic signals, visual signals, auditory signals, magnetic signals, and combinations thereof.

46. A method for rewarding a consumer for a purchase, the method comprising:
determining when the consumer purchases an absorbent product;
providing to the consumer a plurality of choices of food related rewards when the determining indicates that the absorbent product has been purchased by the consumer;
receiving from the consumer a selection of at least one of the food related rewards from the plurality of choices; and
distributing the selection of food related reward to the consumer.

47. The method of claim 46 wherein the absorbent product is selected from the group consisting of diapers, training pants, tampons, napkins, panty liners, interlabial pads, and combinations thereof.

48. The method of claim 46 wherein the absorbent product comprises a unique identifier.

49. The method of claim 48 wherein the unique identifier is selected from the group consisting of an individual bar code, a RFID tag, a product code, a number code, a text code, an alpha-numeric code, and combinations thereof.

50. The method of claim 48 further comprising allowing the consumer to input the unique identifier into a user interface.

51. The method of claim 50 further comprising assigning a point value to the absorbent product.

52. The method of claim 51 further comprising:
identifying the consumer;
accessing a consumer database containing information on previous purchases by the consumer; and
calculating an accumulated point value for all purchases by the consumer;
wherein the plurality of choices of food relate rewards is based upon the consumer's accumulated point value.

53. The method of claim 46 wherein the distributing is selected from the group consisting of providing the consumer with a food benefit coupon, providing a food related item to the consumer, providing a food scented item to the consumer, and combinations thereof.

54. The method of claim 46 wherein the food related reward is delivered to the consumer.

55. The method of claim 46 wherein the food related reward is distributed to the consumer at the time of purchase.

56. The method of claim 46 wherein the food related reward is selected from the group consisting of a food benefit coupon, a food item, a food scented item, and combinations thereof.

57. A system for distributing a reward to a consumer, the system comprising:
means for monitoring consumer purchases to determine when the consumer purchases an absorbent product;
means for signaling a third party when the monitoring means indicate that the absorbent product is purchased by the consumer; and
means for distributing a food related reward to the consumer in response to the purchase of the absorbent product by the consumer.

58. A system for distributing a reward to a consumer, the system comprising:
means for monitoring when the consumer purchases an absorbent product;
means for maintaining a consumer database containing information on monitored purchases by the consumer;
means for receiving from the consumer a selection of a food related reward from a plurality of choices; and
means for distributing the selection of the food related reward to the consumer as function of the information in the consumer database.
